(19) United States
(12) Patent Application Publication
(10) Pub. No.: US 2025/0152072 A1
(43) Pub. Date: May 15, 2025

(54) FEW-SHOT ELECTROCARDIOGRAM (ECG) SIGNAL CLASSIFICATION METHOD BASED ON IMPROVED SIAMESE NETWORK

(71) Applicants: Qilu University of Technology (Shandong Academy of Sciences), Jinan (CN); SHANDONG COMPUTER SCIENCE CENTER (NATIONAL SUPERCOMPUTING CENTER IN JINAN), Jinan (CN)

(72) Inventors: Yinglong WANG, Jinan (CN); Mengyu SUN, Jinan (CN); Minglei SHU, Jinan (CN); Shuwang ZHOU, Jinan (CN); Pengyao XU, Jinan (CN); Zhaoyang LIU, Jinan (CN)

(73) Assignees: Qilu University of Technology (Shandong Academy of Sciences), Jinan (CN); SHANDONG COMPUTER SCIENCE CENTER (NATIONAL SUPERCOMPUTING CENTER IN JINAN), Jinan (CN)

(21) Appl. No.: 18/739,383

(22) Filed: Jun. 11, 2024

(30) Foreign Application Priority Data
Nov. 13, 2023 (CN) 2023114980555

(51) Int. Cl. A61B 5/349; A61B 5/00; A61B 5/308; G16H 50/20

(52) U.S. Cl. CPC A61B 5/349; A61B 5/308; A61B 5/7203; G16H 50/20

(57) ABSTRACT

A few-shot electrocardiogram (ECG) signal classification method based on an improved Siamese network is provided. The method constructs a CMP module as a sub-network of a Siamese network, and combines extracted local and global features to better analyze peak information such as position, amplitude, and offset, making a transformed feature vector more robust. In this way, the method improves the accuracy and stability of few-shot ECG signal classification.

### CROSS-REFERENCE TO THE RELATED APPLICATIONS

This application is based upon and claims priority to Chinese Patent Application No. 202311498055.5, filed on Nov. 13, 2023, the entire contents of which are incorporated herein by reference.

### TECHNICAL FIELD

The present disclosure relates to the technical field of electrocardiogram (ECG) signal classification, and in particular to a few-shot ECG signal classification method based on an improved Siamese network.

### BACKGROUND

In recent years, deep learning (DL)-based algorithm models have achieved unprecedented success in big data (BD) processing in the field of artificial intelligence (AI). However, due to the rarity and large individual differences of certain types of arrhythmias the acquired data is limited, which limits the generalization ability and accuracy of existing models. Few-shot learning is mainly used for neural network classifiers, which only requires a small number of samples for learning and training, and can achieve efficient recognition and classification of electrocardiogram (ECG) signals.

### SUMMARY

In order to overcome the above-mentioned shortcomings in the prior art, the present disclosure provides a few-shot electrocardiogram (ECG) signal classification method based on an improved Siamese network, which can improve the classification accuracy.

In order to solve the technical problem, the present disclosure adopts the following technical solution.

The few-shot ECG signal classification method based on an improved Siamese network includes the following steps:
a) acquiring n original ECG signals to form an original ECG signal set D, D={(x₁, y₁), (x₂, y₂), . . . , (xᵢ, yᵢ), . . . , (xₙ, yₙ)}, where xᵢ denotes an i-th original ECG signal, and yᵢ denotes a class label corresponding to the i-th original ECG signal xᵢ, i∈{1, . . . , n};
b) preprocessing the original ECG signal set D to remove noise in the original ECG signals, thereby acquiring a clean ECG signal set D′, D′={(x′₁, y₁), (x′₂, y₂), . . . , (x′ᵢ, yᵢ), . . . , (x′ₙ, yₙ)}, where x′ᵢ denotes an i-th clean ECG signal;
c) normalizing the i-th clean ECG signal x′ᵢ to acquire a normalized ECG signal x″ᵢ; and performing zero-padding in the end of a sequence of the normalized ECG signal xᵢ″ if a length of the sequence of the normalized ECG signal xᵢ″ is less than Lₘₐₓ, such that the length of the sequence of the normalized ECG signal x″ is equal to Lₘₐₓ, thereby acquiring a normalized ECG signal set D″, D″={(x″₁, y″₁), (x″₂, y₂), . . . , (x″ᵢ, yᵢ), . . . , (x″ₙ, yₙ)}
d) creating a sample pair set P based on the normalized ECG signal set D″,

$$P = \{ ( ( {x}_{1}^{″} , {x}_{2}^{″} ) , {Y}^{′} ) , ( ( {x}_{2}^{″} , {x}_{3}^{″} ) , {Y}^{′} ) , … , ( ( {x}_{i - 1}^{″} , {x}_{i}^{″} ) , {Y}^{′} ) , \\ ( ( {x}_{i}^{″} , {x}_{i + 1}^{″} ) , {Y}^{′} ) , … , ( ( {x}_{n - 2}^{″} , {x}_{n - 1}^{″} ) , {Y}^{′} ) , ( ( {x}_{n - 1}^{″} , {x}_{n}^{″} ) , {Y}^{′} ) \} , where$$

$${Y}^{′} = \{ \begin{matrix}1 & {y}_{i - 1} = {y}_{i} \\ 0 & {y}_{i - 1} \neq {y}_{i}\end{matrix} ;$$

yᵢ₋₁ denotes a class label corresponding to the (i−1)-th original ECG signal xᵢ₋₁; and there are M sample pairs in the sample pair set P,

$$M = \frac{n \times ( n - 1 )}{2} ;$$

e) constructing a few-shot classification model, and inputting a sample pair ((x″₁, x″ᵢ₊₁), Y′) from the sample pair set P into the few-shot classification model to acquire a similarity score E_{w}(x″ᵢ, x″ᵢ₊₁);
f) training, by an adaptive moment estimation (Adam) optimizer, the few-shot classification model through a loss function L to acquire an optimized few-shot classification model;
g) randomly sampling K ECG signals from each of N classes in a Massachusetts Institute of Technology-Beth Israel Hospital (MIT-BIH) dataset to form a support set sₛᵤₚₚₒᵣₜ, sₛᵤₚₚₒᵣₜ={(s₁, a₁), (s₂, a₂), . . . , (sᵢ, aᵢ), . . . , (s_{NK}, a_{NK})}, where sᵢ denotes an i-th ECG signal, and aᵢ denotes a class label corresponding to the i-th ECG signal sᵢ, i∈{1, . . . , NK};
h) randomly sampling Q ECG signals from each of the N classes in the MIT-BIH dataset to form a query set s_{query}, s_{query}={(q₁, b₁), (q₂, b₂), . . . , (qᵢ, bᵢ), . . . , (q_{NQ}, b_{NQ})}, where qᵢ denotes an i-th ECG signal, and b; denotes a class label corresponding to the i-th ECG signal qᵢ, i∈{1, . . . , NQ};
i) replacing the i-th original ECG signal xᵢ with the i-th ECG signal sᵢ, and repeating the steps b) and c) to acquire an i-th normalized ECG signal s″ᵢ, thereby acquiring a normalized support set s′ₛᵤₚₚₒᵣₜ, s″ₛᵤₚₚₒᵣₜ={(s″₁, a₁), (s″₂, a₂), . . . , (s″ᵢ, aᵢ), . . . , (s′_{NK}, a_{NK})}; and replacing the i-th original ECG signal xᵢ with the i-th ECG signal qᵢ, and repeating the steps b) and c) to acquire an i-th normalized ECG signal q″ᵢ, thereby acquiring a normalized query set s″_{query}, s″_{query}={(q″₁, b₁), (q″₂, b₂), . . . , (q″ᵢ, bᵢ), . . . , (q″_{NQ}, b_{NQ})}; and
j) inputting the i-th normalized ECG signal s″ᵢ and the i-th normalized ECG signal q″ᵢ into the optimized few-shot classification model to acquire a classification result.

Further, the step a) includes: acquiring the n original ECG signals from a University of California Riverside (UCR) dataset.

Further, the step b) includes: denoising, by a first median filter and a second median filter in sequence, the i-th original ECG signal xᵢ to acquire the i-th clean ECG signal x′ᵢ.

Preferably, the first median filter has a width of 300 ms, and the second median filter has a width of 600 ms.

Preferably, Lₘₐₓ=187.

Further, the step e) includes:
e-1) constructing the few-shot classification model, including an embedding module and a metric module;
e-2) constructing the embedding module of the few-shot classification model, where the embedding module includes a Siamese network formed by a first CMP module and a second CMP module; the first CMP module includes a convolutional layer, a first rectified linear unit (ReLU) activation function layer, a primary capsule layer of a capsule network, a digital capsule layer of the capsule network, a first fully connected layer, a second ReLU activation function layer, and a second fully connected layer; and the second CMP module includes a convolutional layer, a first ReLU activation function layer, a primary capsule layer of a capsule network, a digital capsule layer of the capsule network, a first fully connected layer, a second ReLU activation function layer, and a second fully connected layer;
e-3) inputting the i-th normalized ECG signal x″₁ into the convolutional layer and the first ReLU activation function layer of the first CMP module in sequence to acquire a feature f₁¹; inputting the feature f₁¹ into the primary capsule layer of the capsule network in the first CMP module to acquire a vector f₁²; inputting the vector f₁² into the digital capsule layer of the capsule network in the first CMP module to acquire a feature f₁³; inputting the feature f₁³ into the first fully connected layer and the second ReLU activation function layer of the first CMP module in sequence to acquire a feature f₁⁴; and inputting the feature f₁⁴ into the second fully connected layer of the first CMP module to acquire a feature f(x″ᵢ);
e-4) inputting the (i+1)-th normalized ECG signal x″ᵢ₊₁ into the convolutional layer and the first ReLU activation function layer of the first CMP module in sequence to acquire a feature f₂¹; inputting the feature f₂¹ into the primary capsule layer of the capsule network in the first CMP module to acquire a vector f₂²; inputting the vector f₂² into the digital capsule layer of the capsule network in the first CMP module to acquire a feature f₂³; inputting the feature f₂³ into the first fully connected layer and the second ReLU activation function layer of the first CMP module in sequence to acquire a feature f₂⁴; and inputting the feature f₂⁴ into the second fully connected layer of the first CMP module to acquire a feature f(x″₁₊₁); and
e-5) inputting the feature f(x″ᵢ) and the feature f(x″ᵢ₊₁) into the metric module of the few-shot classification model, and calculating the similarity score E_{w}(x″₁, x″ᵢ₊₁) by E_{w}(x″ᵢ, x″ᵢ₊₁)=∥f(x″ᵢ)−f(x″ᵢ₊₁)∥, where ∥●∥ denotes a Euclidean distance (ED) calculation.

Preferably, in the step e-2), the convolutional layer of the first CMP module includes a 3×3 convolution kernel, and the convolutional layer of the second CMP module includes a 3×3 convolution kernel.

Further, the step f) includes: calculating the loss function

$$L by L = {L}_{1} + α {L}_{2} ,$$

$$where {L}_{1} = {Y}^{′} \frac{1}{2} {( {E}_{w} ( {x}_{i}^{″} , {x}_{i + 1}^{″} ) )}^{2} + ( 1 - {Y}^{′} ) {\{ max ( 0 , m - {E}_{w} ( {x}_{i}^{″} , {x}_{i + 1}^{″} ) ) \}}^{2} ;$$

m denotes a hyperparameter, α denotes a hyperparameter; and L₂ denotes a cross entropy loss function.

Further, the step j) includes:
j-1) inputting the i-th normalized ECG signal s″ᵢ of a u-th class into the convolutional layer and the first ReLU activation function layer of the first CMP module in sequence to acquire a feature f₃¹, u∈{1, . . . , N}; inputting the feature f₃¹ into the primary capsule layer of the capsule network in the first CMP module to acquire a vector f₃²; inputting the vector f₃² into the primary capsule layer of the capsule network in the first CMP module to acquire a feature f₃³; inputting the feature f₃³ into the first fully connected layer and the second ReLU activation function layer of the first CMP module in sequence to acquire a feature f₃⁴; inputting the feature f₃⁴ into the second fully connected layer of the first CMP module to acquire a feature f(s″ᵢ)ᵤ; and calculating, by a mean( ) function in Python, an average of all K features f(s″₁)ᵤ, f(s″₂)ᵤ, . . . , f(s″ᵢ)ᵤ, . . . , f(s″_{K})ᵤ, of the u-th class to acquire a feature vector μᵤ;
j-2) inputting the i-th normalized ECG signal q″ᵢ into the convolutional layer and the first ReLU activation function layer of the first CMP module in sequence to acquire a feature f₄¹; inputting the feature f₄¹ into the primary capsule layer of the capsule network in the first CMP module to acquire a vector f₄²; inputting the vector f₄² into the primary capsule layer of the capsule network in the first CMP module to acquire a feature f₄³; inputting the feature f₄³ into the first fully connected layer and the second ReLU activation function layer of the first CMP module in sequence to acquire a feature f₄⁴; and inputting the feature f₄⁴ into the second fully connected layer of the first CMP module to acquire a feature f(q″ᵢ);
j-3) inputting the feature vector μᵤ and the feature f(q″ᵢ) into the metric module of the few-shot classification model, and calculating the similarity score E_{w}(μᵤ, f(q″ᵢ)) by E_{w}(μᵤ, f(q″ᵢ))=∥μᵤ−f(q″ᵢ)∥; and
j-4) calculating a class label ŷᵢ of the i-th normalized ECG signal q″ᵢ by \vec{y}ᵢ=arg max {E_{w}(μ₁, f(q″ᵢ)), E_{w}(μ₂, f(q″ᵢ)), . . . , E_{w}(μᵤ, f(q″ᵢ)), . . . , E_{w}(μ_{N}, f(q″ᵢ))}, and combining class labels of all NQ normalized ECG signals to form the classification result.

The present disclosure has the following beneficial effects. The present disclosure constructs the CMP module as a sub-network of the Siamese network, and combines the extracted local and global features to better analyze peak information such as position, amplitude, and offset, making the transformed feature vector more robust. In this way, the present disclosure improves the accuracy and stability of few-shot ECG signal classification.

### BRIEF DESCRIPTION OF THE DRAWINGS

FIG. 1 is a flowchart of a few-shot ECG signal classification method based on an improved Siamese network according to the present disclosure;

FIG. 2 is a structural diagram of a CMP module according to the present disclosure;

FIG. 3 shows a comparison of average accuracy and K for different models according to the present disclosure;

FIGS. 4A-4B show a comparison of confusion matrices for models in 3-way 10-shot according to the present disclosure; and

FIGS. 5A-5F show a comparison between true and predict labels in 3-way 10-shot.

Table 1 Average accuracy comparison results of models in the present disclosure

Table 2 Average precision, average recall, and average F1 score comparison results of different models in the present disclosure

### DETAILED DESCRIPTION OF THE EMBODIMENTS

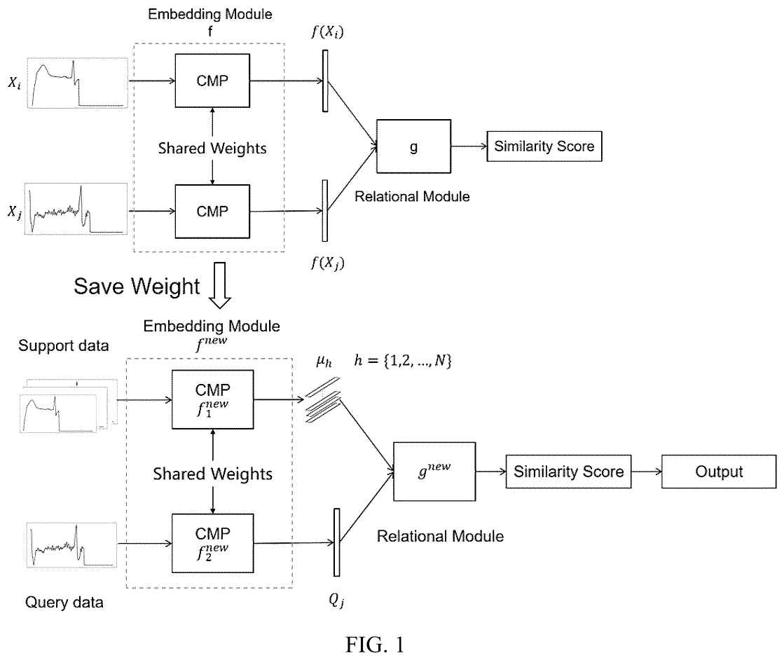

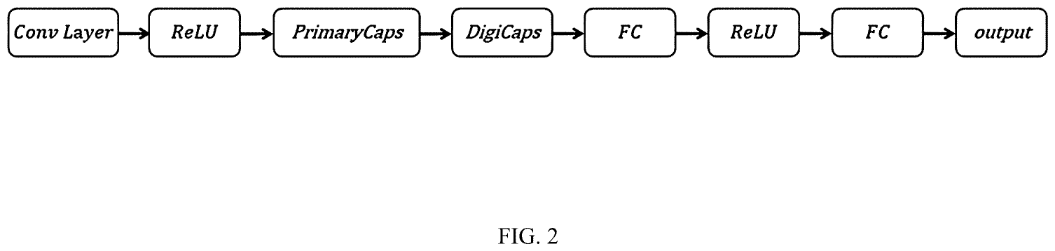

The present disclosure is further described with reference to FIG. 1 and FIG. 2.

The few-shot ECG signal classification method based on an improved Siamese network includes the following steps:
a) n original ECG signals are acquired to form original ECG signal set D, D={(x₁, y₁), (x₂, y₂), . . . , (xᵢ, yᵢ), . . . , (xₙ, yₙ)}, where xᵢ denotes i-th original ECG signal, and yᵢ denotes a class label corresponding to the i-th original ECG signal xᵢ, i∈{1, . . . , n}.
b) The original ECG signal set D is preprocessed to remove noise in the original ECG signals, thereby acquiring clean ECG signal set D′, D′={(x′₁, y₁), (x′₂, y₂), . . . , (x′ᵢ, yᵢ), . . . , (x′ₙ, yₙ)}, where x′ᵢ denotes i-th clean ECG signal.
c) The i-th clean ECG signal x′ᵢ is normalized to acquire normalized ECG signal x″ᵢ; and performing zero-padding is performed in the end of a sequence of the normalized ECG signal x″ᵢ if a length of the sequence of the normalized ECG signal x″ᵢ is less than Lₘₐₓ, such that the length of the sequence of the normalized ECG signal x″ᵢ is equal to Lₘₐₓ, thereby acquiring normalized ECG signal set D″, D″={x″₁, y₁), (x″₂, y₂), . . . , (x″ᵢ, yᵢ), . . . , (x″ₙ, yₙ)}.
d) Sample pair set P is created based on the normalized ECG signal set D″,

$$P = \{ ( ( {x}_{1}^{″} , {x}_{2}^{″} ) , {Y}^{′} ) , ( ( {x}_{2}^{″} , {x}_{3}^{″} ) , {Y}^{′} ) , … , ( ( {x}_{i - 1}^{″} , {x}_{i}^{″} ) , {Y}^{′} ) , \\ ( ( {x}_{i}^{″} , {x}_{i + 1}^{″} ) , {Y}^{′} ) , … , ( ( {x}_{n - 2}^{″} , {x}_{n - 1}^{″} ) , {Y}^{′} ) , ( ( {x}_{n - 1}^{″} , {x}_{n}^{″} ) , {Y}^{′} ) \} , where$$

$${Y}^{′} = \{ \begin{matrix}1 & {y}_{i - 1} = {y}_{i} \\ 0 & {y}_{i - 1} \neq {y}_{i}\end{matrix} ;$$

yᵢ₋₁ denotes a class label corresponding to the (i−1)-th original ECG signal x₁₋₁; and there are M sample pairs in the sample pair set P,

$$M = \frac{n \times ( n - 1 )}{2} ;$$

e) A few-shot classification model is constructed, and sample pair (x″ᵢ, x″₁₋₁),Y″) from the sample pair set P is input into the few-shot classification model to acquire similarity score E_{w}(x″ᵢ, x″ᵢ₊₁)
f) The few-shot classification model is trained by an adaptive moment estimation (Adam) optimizer through loss function L to acquire an optimized few-shot classification model.
g) K ECG signals are randomly sampled from each of N classes in a Massachusetts Institute of Technology-Beth Israel Hospital (MIT-BIH) dataset to form support set sₛᵤₚₚₒᵣₜ, sₛᵤₚₚₒᵣₜ={(s₁, a₁), (s₂, a₂), . . . , (sᵢ, aᵢ), . . . , (s_{NK}, a_{NK})}, where sᵢ denotes i-th ECG signal, and aᵢ denotes a class label corresponding to the i-th ECG signal sᵢ, i∈{1, . . . , NK}.
h) Q ECG signals are randomly sampled from each of the N classes in the MIT-BIH dataset to form query set s_{query} for the purpose of accurately classifying NQ queries based on given NK samples, s_{query}={(q₁, b₁), (q₂, b₂), . . . , (qᵢ, bᵢ), . . . , (q_{NQ}, b_{NQ})}, where qᵢ denotes i-th ECG signal, and b; denotes a class label corresponding to the i-th ECG signal qᵢ, i∈{1, . . . , NQ}.

i) The i-th original ECG signal xᵢ is replaced with the i-th ECG signal sᵢ, and the steps b) and c) are repeated to acquire i-th normalized ECG signal s″ᵢ, thereby acquiring normalized support set s″ₛᵤₚₚₒᵣₜ, s″ₛᵤₚₚₒᵣₜ={(s″₁, a₁), (s″₂, a₂), . . . , (s″ᵢ, aᵢ), . . . , (s″_{NK}, a_{NK})}. The i-th original ECG signal xᵢ is replaced with the i-th ECG signal qᵢ, and the steps b) and c) are repeated to acquire i-th normalized ECG signal q″ᵢ, thereby acquiring normalized query set query s″_{query}, s″_{query}={(q″₁, b₁), (q″₂, b₂), . . . , (q″ᵢ, bᵢ), . . . , (q″_{NQ}, B_{NQ})}.
j) The i-th normalized ECG signal s″ᵢ and the i-th normalized ECG signal q″ᵢ are input into the optimized few-shot classification model to acquire classification result.

The present disclosure provides a brand new CMP module to establish the Siamese network for few-shot ECG signal classification, which improves classification accuracy.

In an embodiment of the present disclosure, in the step a), the n original ECG signals are acquired from a University of California Riverside (UCR) dataset.

In an embodiment of the present disclosure, in the step b), the i-th original ECG signal xᵢ is denoised by a first median filter and a second median filter in sequence to acquire the i-th clean ECG signal x′ᵢ. In the embodiment, preferably, the first median filter has a width of 300 ms, and the second median filter has a width of 600 ms.

In an embodiment of the present disclosure, Lₘₐₓ=187.

In an embodiment of the present application, the step e) is as follows.
e-1) The few-shot classification model is constructed, including an embedding module and a metric module.
e-2) The embedding module of the few-shot classification model is constructed, where the embedding module includes a Siamese network formed by a first CMP module and a second CMP module; the first CMP module includes a convolutional layer, a first rectified linear unit (ReLU) activation function layer, a primary capsule layer of a capsule network, a digital capsule layer of the capsule network, a first fully connected layer, a second ReLU activation function layer, and a second fully connected layer; and the second CMP module includes a convolutional layer, a first ReLU activation function layer, a primary capsule layer of a capsule network, a digital capsule layer of the capsule network, a first fully connected layer, a second ReLU activation function layer, and a second fully connected layer.
e-3) The i-th normalized ECG signal x″ᵢ is input into the convolutional layer and the first ReLU activation function layer of the first CMP module in sequence to extract low-level feature of the ECG signal x″ᵢ, thereby acquiring feature f₁¹. The feature f₁¹ is input into the primary capsule layer of the capsule network in the first CMP module for a feature-to-vector transformation, thereby acquiring vector f₁². The vector f₁² is input into the digital capsule layer of the capsule network in the first CMP module, and the vector f₁² is subjected to matrix transformation, input weighting, summation, and non-linear transformation to acquire feature f₁³. The feature f₃¹ is input into the zero-neuron first fully connected layer and second ReLU activation function layer of the first CMP module in sequence for nonlinear mapping to acquire feature f₁⁴. The feature f₁⁴ is input into the second fully connected layer of the first CMP module, and an embedding vector mapped from the first fully connected layer to a 0-dimensional space outputs an embedding vector with a same dimension as an input dimension to acquire feature f(x″ᵢ).
e-4) The (i+1)-th normalized ECG signal x″ᵢ₊₁ is input into the convolutional layer and the first ReLU activation function layer of the first CMP module in sequence to extract low-level feature of the ECG signal x″ᵢ₊₁, thereby acquiring feature f₂¹. The feature f₂¹ is input into the primary capsule layer of the capsule network in the first CMP module for a feature-to-vector transformation, thereby acquiring vector f₂². The vector f₂² is input into the digital capsule layer of the capsule network in the first CMP module, and the vector f₂² is subjected to matrix transformation, input weighting, summation, and non-linear transformation to acquire feature f₂³. The feature f₂³ is input into the zero-neuron first fully connected layer and second ReLU activation function layer of the first CMP module in sequence for nonlinear mapping to acquire feature f₂⁴. The feature f₂⁴ is input into the second fully connected layer of the first CMP module, and an embedding vector mapped from the first fully connected layer to a 0-dimensional space outputs an embedding vector with a same dimension as an input dimension to acquire feature f(x″ᵢ₊₁₎.
e-5) The feature f(x″ᵢ) and the feature f(x″ᵢ₊₁) are input into the metric module of the few-shot classification model, and the similarity score is E_{w}(x″ᵢ, x″ᵢ₊₁) calculated by E_{w}(x″₁, x″ᵢ₊₁)=∥f(x″ᵢ)−f(x″ᵢ₊₁)∥, where ∥●∥ denotes a Euclidean distance (ED) calculation.

In the embodiment, in the step e-2), the convolutional layer of the first CMP module includes a 3×3 convolution kernel, and the convolutional layer of the second CMP module includes a 3×3 convolution kernel.

In the step f), the loss function L is calculated by L=L₁+αL₂, where L₁ is designed to adjust the loss function of the Siamese network.

$${L}_{1} = {Y}^{′} \frac{1}{2} {( {E}_{w} ( {x}_{i}^{″} , {x}_{i + 1}^{″} ) )}^{2} + ( 1 - {Y}^{′} ) {\{ max ( 0 , m - {E}_{w} ( {x}_{i}^{″} , {x}_{i + 1}^{″} ) ) \}}^{2} ,$$

where m denotes a hyperparameter; α denotes a hyperparameter; and L₂ denotes a cross entropy loss function. Further, α=5, m=5. The total loss L takes into account both sample distance and feature classification.

The step j) is as follows.
j-1) The i-th normalized ECG signal s″ᵢ of a W-th class is input into the convolutional layer and the first ReLU activation function layer of the first CMP module in sequence to acquire feature f₃¹, u∈{1, . . . , N}. The feature f₃¹ is input into the primary capsule layer of the capsule network in the first CMP module to acquire vector f₃². The vector f₃² is input into the primary capsule layer of the capsule network in the first CMP module to acquire feature f₃³. The feature f₃³ is input into the first fully connected layer and the second ReLU activation function layer of the first CMP module in sequence to acquire feature f₃⁴. The feature f₃⁴ is input into the second fully connected layer of the first CMP module to acquire feature f(s″ᵢ)ᵤ. An average of all K features f(s″₁)ᵤ, f(s″₂)ᵤ, . . . , f(s″ᵢ), . . . , f(s″_{K})ᵤ of the W-th class is calculated by a mean( ) function in Python to acquire feature vector μᵤ.
j-2) The i-th normalized ECG signal q″ᵢ is input into the convolutional layer and the first ReLU activation function layer of the first CMP module in sequence to acquire feature f₄¹. The feature f₄¹ is input into the primary capsule layer of the capsule network in the first CMP module to acquire vector f₄². The vector f₄² is input into the primary capsule layer of the capsule network in the first CMP module to acquire feature f₄³. The feature f₄³ is input into the first fully connected layer and the second ReLU activation function layer of the first CMP module in sequence to acquire feature f₄⁴. The feature f₄⁴ is input into the second fully connected layer of the first CMP module to acquire feature f(q″ᵢ).
j-3) The feature vector μᵤ and the feature f(q″ᵢ) are input into the metric module of the few-shot classification model, and the similarity score is E_{w}(μᵤ, f(q″ᵢ)) is calculated by E_{w}(μᵤ, f(q″ᵢ))=∥μᵤ−f(q″ᵢ)∥.
j-4) Class label ŷᵢ of the i-th normalized ECG signal q″ᵢ is calculated by \vec{y}ᵢ=arg max {E_{w}(μ₁, f(q″ᵢ)), E_{w}(μ₂, f(q″ᵢ)), . . . , E_{w}(μᵤ, f(q″ᵢ)), . . . , E_{w}(μ_{N}, f(q″ᵢ))}, and class labels of all NQ normalized ECG signals are combined in to the classification result.

Taking the publicly available MIT-BIH dataset as an example, the implementation of the present disclosure is explained in detail below.

The model proposed by the present disclosure is compared with mainstream classification task models (ED, dynamic time warping (DTW), long short-term memory-fully connected network (LSTM-FCN)) and a Siamese convolutional neural network (SCNN) model, and the final accuracy is the average of 20 tasks. Accuracy, precision, recall, and F1 score are used as evaluation indicators.

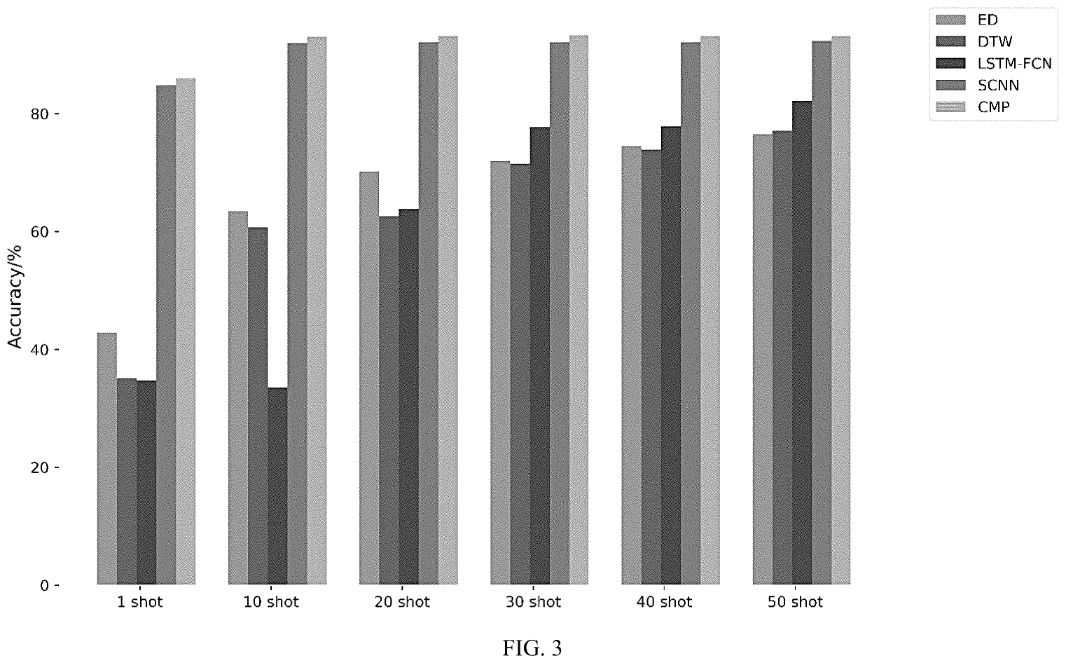

The training is performed based on UCR ECG200 and ECG5000 datasets, the validation is performed based on UCR TwoLeadECG and ECGFiveDays datasets, and the model testing is performed based on the MIT-BIH dataset. FIG. 3 shows a comparison of the relationship between the average accuracy and K for different models. It can be seen from the figure that as K increases, ED almost monotonically increases, and the precision, recall, and F1 score also increase. DTW does not follow such a smooth behavior and offers poorer performance than ED at a smaller K value. However, DTW outperforms ED at a value close to 50 and may perform better at a larger value. Unlike ED and DTW, FCN-LSTM exhibits an extremely irregular behavior during training, with a significant fluctuation in accuracy in certain areas, which can be attributed to the randomness of neural network optimization and the lack of labeled data for training. The comparison between the model of the present disclosure and the SCNN model shows that the accuracy does not increase sharply from K=1 to K=50, but tends to stabilize around 0.93, and the recall, precision, and F1 score also tend to stabilize around 0.93.

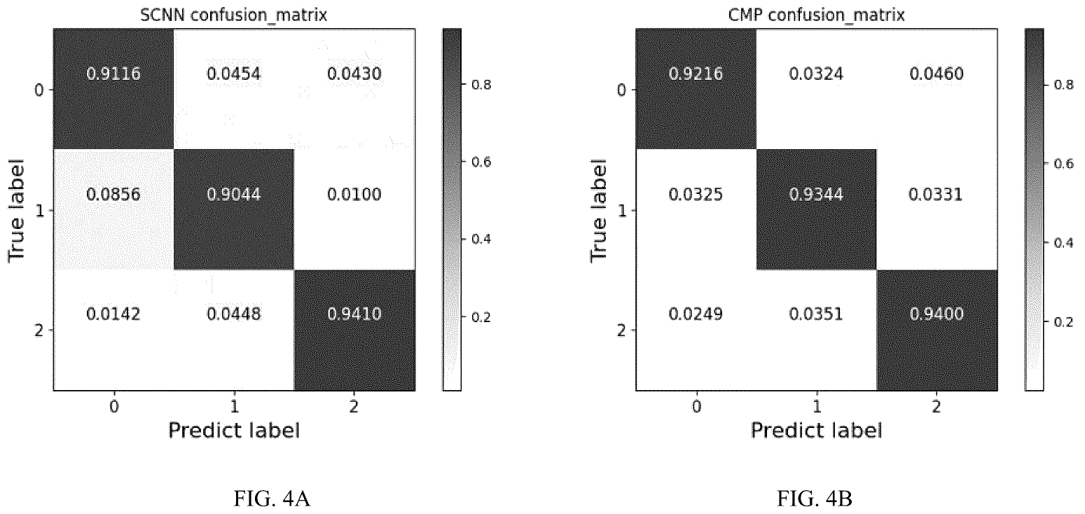

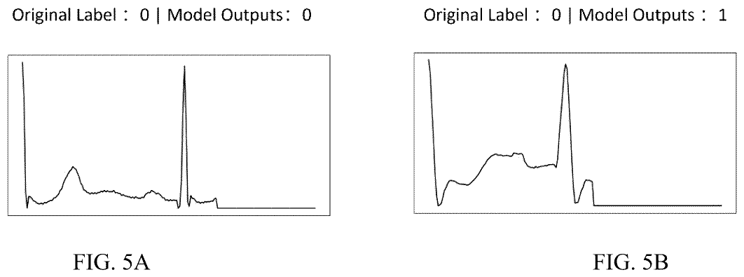

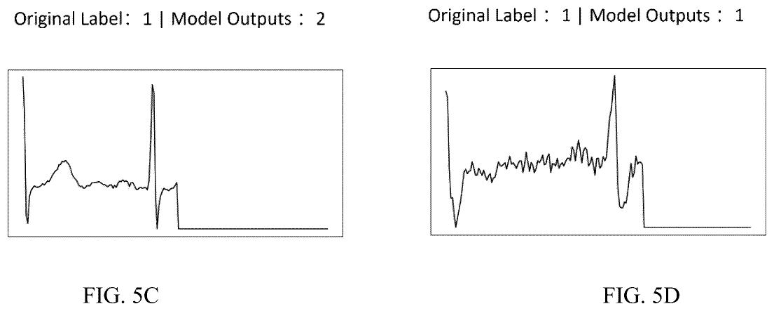

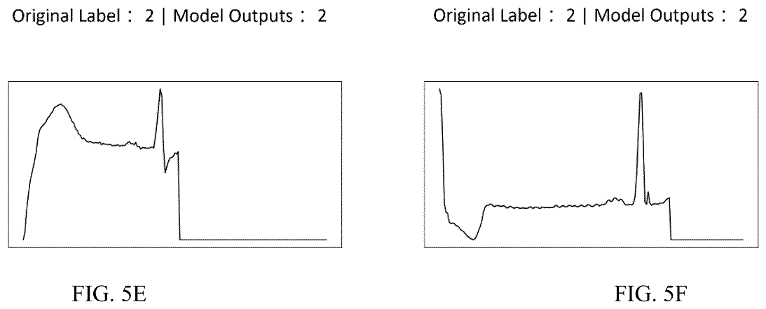

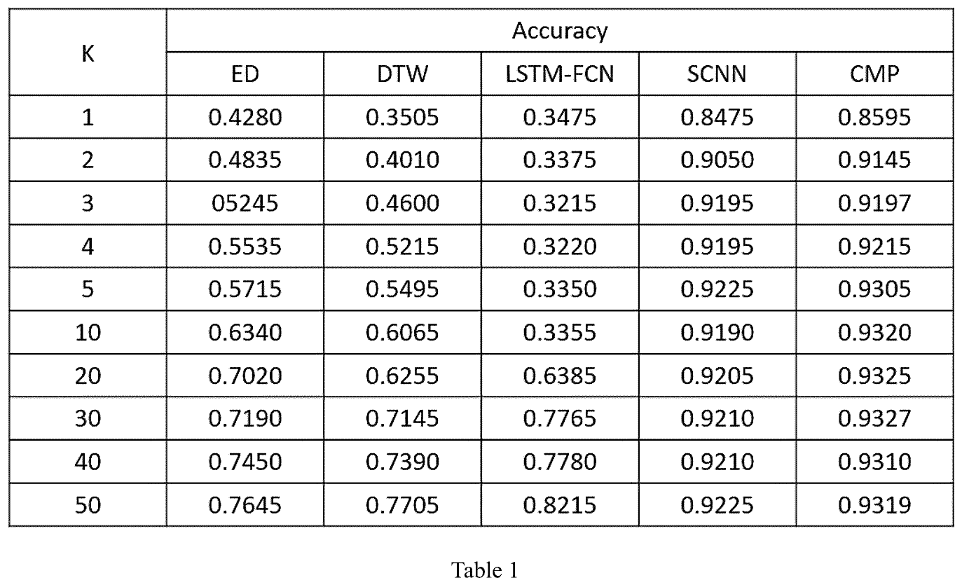

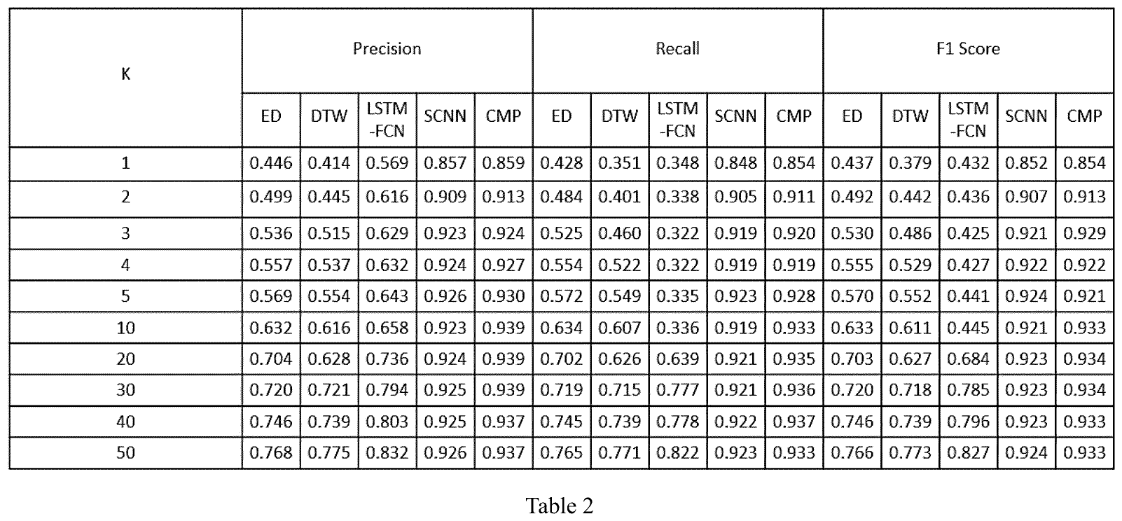

FIGS. 4A-4B show a confusion matrix of the CMP model in 3-way 10-shot on the MIT-BIH dataset. It can be seen from the figure that the model of the present disclosure has better comprehensive performance and lower misdiagnosis rate during the evaluation process. FIGS. 5A-5F show changes in true and predict labels of 6 randomly selected signals during 3-way 10-shot (N, S and V are represented by 0, 1 and 2, respectively). Table 1 shows comparison results of accuracy acquired by different models under different K values on the MIT-BIH dataset, while Table 2 shows comparison results of average precision, average recall, and average F1 score of different models on the MIT-BIH dataset. In summary, from the perspective of model performance, the model of the present disclosure can effectively distinguish between acceptable and unacceptable ECG signals in practical environments.

Finally, it should be noted that the above descriptions are only preferred embodiments of the present disclosure, and are not intended to limit the present disclosure. Although the present disclosure has been described in detail with reference to the foregoing embodiments, those skilled in the art may still modify the technical solutions described in the foregoing embodiments, or equivalently substitute some technical features thereof. Any modification, equivalent substitution, improvement, etc. within the spirit and principles of the present disclosure shall fall within the scope of protection of the present disclosure.

What is claimed is:

1. A few-shot electrocardiogram (ECG) signal classification method based on an improved Siamese network, comprising the following steps:
a) acquiring n original ECG signals to form an original ECG signal set D, D={(x₁, y₁), (x₂, y₂), . . . , (xᵢ, yᵢ), . . . , (xₙ, yₙ)}, wherein xᵢ denotes an i-th original ECG signal, and yᵢ denotes a class label corresponding to the i-th original ECG signal xᵢ, i∈{1, . . . , n};
b) preprocessing the original ECG signal set D to remove noise in the n original ECG signals, thereby acquiring a clean ECG signal set D′, D′={(x′₁, y₁), (x′₂, y₂), . . . , (x′ᵢ, yᵢ), . . . , (x′ₙ, yₙ)}, wherein x′ᵢ denotes an i-th clean ECG signal;
c) normalizing the i-th clean ECG signal x′ᵢ to acquire a normalized ECG signal x″ᵢ; and performing zero-padding in an end of a sequence of the normalized ECG signal x″ᵢ if a length of the sequence of the normalized ECG signal x″ᵢ is less than Lₘₐₓ, wherein the length of the sequence of the normalized ECG signal x″ᵢ is equal to Lₘₐₓ, and a normalized ECG signal set D″ is acquired, D″={(x″₁, y₁), (x″₂, y₂), . . . , (x″ᵢ, yᵢ), . . . , (x″ᵢ, yᵢ)};
d) creating a sample pair set P based on the normalized ECG signal set D″,
$$P = \{ ( ( {x}_{1}^{″} , {x}_{2}^{″} ) , {Y}^{′} ) , ( ( {x}_{2}^{″} , {x}_{3}^{″} ) , {Y}^{′} ) , … , ( ( {x}_{i - 1}^{″} , {x}_{i}^{″} ) , {Y}^{′} ) , \\ ( ( {x}_{i}^{″} , {x}_{i + 1}^{″} ) , {Y}^{′} ) , … , ( ( {x}_{n - 2}^{″} , {x}_{n - 1}^{″} ) , {Y}^{′} ) , ( ( {x}_{n - 1}^{″} , {x}_{n}^{″} ) , {Y}^{′} ) \} , wherein$$
$${Y}^{′} = \{ \begin{matrix}1 & {y}_{i - 1} = {y}_{i} \\ 0 & {y}_{i - 1} \neq {y}_{i}\end{matrix} ;$$
yᵢ₋₁ denotes a class label corresponding to an (i−1)-th original ECG signal xᵢ₋₁; and there are M sample pairs in the sample pair set P,
$$M = \frac{n \times ( n - 1 )}{2} ;$$
e) constructing a few-shot classification model, and inputting a sample pair ((x′ᵢ, x′ᵢ₊₁), Y′) from the sample pair set P into the few-shot classification model to acquire a similarity score E_{w}(x″ᵢ, x″ᵢ₊₁);
f) training, by an adaptive moment estimation (Adam) optimizer, the few-shot classification model through a loss function L to acquire an optimized few-shot classification model;
g) randomly sampling K ECG signals from each of N classes in a Massachusetts Institute of Technology-Beth Israel Hospital (MIT-BIH) dataset to form a support set sₛᵤₚₚₒᵣₜ, sₛᵤₚₚₒᵣₜ={(s₁, a₁), (s₂, a₂), . . . , (sᵢ, aᵢ), . . . , (s_{NK}, a_{NK})}, wherein S; denotes an i-th ECG signal, and aᵢ denotes a class label corresponding to the i-th ECG signal sᵢ, i∈{1, . . . , NK};
h) randomly sampling Q ECG signals from each of the N classes in the MIT-BIH dataset to form a set s_{query}, s_{query}={(q₁, b₁), (q₂, b₂), . . . , (qᵢ, bᵢ), . . . , (q_{NQ}, b_{NQ})}, wherein qᵢ denotes an i-th ECG signal, and bᵢ denotes a class label corresponding to the i-th ECG signal qᵢ, i∈{1, . . . , NQ};
i) replacing the i-th original ECG signal xᵢ with the i-th ECG signal sᵢ, and repeating the steps b) and c) to acquire an i-th normalized ECG signal s″ᵢ, wherein a normalized support set s″ₛᵤₚₚₒᵣₜ is acquired, s″ₛᵤₚₚₒᵣₜ={(s″₁, a₁), (s″₂, a₂), . . . , (s″ᵢ, aᵢ), . . . , (s″_{NK}, a_{NK})}; and replacing the i-th original ECG signal xᵢ with the i-th ECG signal qᵢ, and repeating the steps b) and c) to acquire an i-th normalized ECG signal q″ᵢ, a normalized query set s″_{query} is acquired, s′_{query}={(q″₁, b₁), (q″₂, b₂), . . . , (a″ᵢ, bᵢ), . . . , (q″_{NQ}, b_{NQ})}; and
j) inputting the i-th normalized ECG signal s″ᵢ and the i-th normalized ECG signal q″ᵢ into the optimized few-shot classification model to acquire a classification result.

2. The few-shot ECG signal classification method based on the improved Siamese network according to claim 1, wherein the step a) comprises: acquiring the n original ECG signals from a University of California Riverside (UCR) dataset.

3. The few-shot ECG signal classification method based on the improved Siamese network according to claim 1, wherein the step b) comprises: denoising, by a first median filter and a second median filter in sequence, the i-th original ECG signal xᵢ to acquire the i-th clean ECG signal x′ᵢ.

4. The few-shot ECG signal classification method based on the improved Siamese network according to claim 3, wherein the first median filter has a width of 300 ms, and the second median filter has a width of 600 ms.

5. The few-shot ECG signal classification method based on the improved Siamese network according to claim 1, wherein Lₘₐₓ=187.

6. The few-shot ECG signal classification method based on the improved Siamese network according to claim 1, wherein the step e) comprises:
e-1) constructing the few-shot classification model, comprising an embedding module and a metric module;
e-2) constructing the embedding module of the few-shot classification model, wherein the embedding module comprises a Siamese network formed by a first CMP module and a second CMP module; the first CMP module comprises a convolutional layer, a first rectified linear unit (ReLU) activation function layer, a primary capsule layer of a capsule network, a digital capsule layer of the capsule network, a first fully connected layer, a second ReLU activation function layer, and a second fully connected layer; and the second CMP module comprises a convolutional layer, a first ReLU activation function layer, a primary capsule layer of a capsule network, a digital capsule layer of the capsule network, a first fully connected layer, a second ReLU activation function layer, and a second fully connected layer;
e-3) inputting the i-th normalized ECG signal x″ᵢ into the convolutional layer and the first ReLU activation function layer of the first CMP module in sequence to acquire a feature f₁¹; inputting the feature f₁¹ into the primary capsule layer of the capsule network in the first CMP module to acquire a vector f₁²; inputting the vector f₁² into the digital capsule layer of the capsule network in the first CMP module to acquire a feature f₁³; inputting the feature f₁³ into the first fully connected layer and the second ReLU activation function layer of the first CMP module in sequence to acquire a feature f₁⁴; and inputting the feature f₁⁴ into the second fully connected layer of the first CMP module to acquire a feature f(x″ᵢ);
e-4) inputting an (i+1)-th normalized ECG signal x″ᵢ₊₁ into the convolutional layer and the first ReLU activation function layer of the first CMP module in sequence to acquire a feature f₂¹; inputting the feature f₂¹ into the primary capsule layer of the capsule network in the first CMP module to acquire a vector f₂²; inputting the vector f₂² into the digital capsule layer of the capsule network in the first CMP module to acquire a feature inputting the feature f₂³ into the first fully connected layer and the second ReLU activation function layer of the first CMP module in sequence to acquire a feature f₂⁴; and inputting the feature f₂⁴ into the second fully connected layer of the first CMP module to acquire a feature f(x″ᵢ₊₁); and
e-5) inputting the feature f(x″ᵢ) and the feature f(x″ᵢ₊₁) into the metric module of the few-shot classification model, and calculating the similarity score E_{w}(x″₁, x″ᵢ₊₁) by E_{w}(x″ᵢ, x″ᵢ₊₁)=∥f(x″ᵢ)−f(x″ᵢ₊₁)∥, wherein ∥●∥ denotes a Euclidean distance (ED) calculation.

7. The few-shot ECG signal classification method based on the improved Siamese network according to claim 6, wherein in the step e-2), the convolutional layer of the first CMP module comprises a 3×3 convolution kernel, and the convolutional layer of the second CMP module comprises a 3×3 convolution kernel.

8. The few-shot ECG signal classification method based on the improved Siamese network according to claim 1, wherein the step f) comprises: calculating the loss function L by, L=L₁+αL₂, wherein
$${L}_{1} = {Y}^{′} \frac{1}{2} {( {E}_{w} ( {x}_{i}^{″} , {x}_{i + 1}^{″} ) )}^{2} + ( 1 - {Y}^{′} ) {\{ max ( 0 , m - {E}_{w} ( {x}_{i}^{″} , {x}_{i + 1}^{″} ) ) \}}^{2} ;$$
m denotes a hyperparameter, α denotes a hyperparameter; and L₂ denotes a cross entropy loss function.

9. The few-shot ECG signal classification method based on the improved Siamese network according to claim 6, wherein the step j) comprises:
j-1) inputting the i-th normalized ECG signal s″ᵢ of a u-th class into the convolutional layer and the first ReLU activation function layer of the first CMP module in sequence to acquire a feature f₃¹, u∈{1, . . . , N}; inputting the feature f₃¹ into the primary capsule layer of the capsule network in the first CMP module to acquire a vector f₃²; inputting the vector f₃² into the primary capsule layer of the capsule network in the first CMP module to acquire a feature f₃³; inputting the feature f₃³ into the first fully connected layer and the second ReLU activation function layer of the first CMP module in sequence to acquire a feature f₃⁴; inputting the feature f₃⁴ into the second fully connected layer of the first CMP module to acquire a feature f(s″ᵢ)ᵤ; and calculating, by a mean( ) function in Python, an average of all K features f(s″₁)ᵤ, f(s″₂)ᵤ, . . . , f(s″ᵢ)ᵤ, . . . , f(s″_{K})ᵤ, of the u-th class to acquire a feature vector μᵤ;
j-2) inputting the i-th normalized ECG signal q″ᵢ into the convolutional layer and the first ReLU activation function layer of the first CMP module in sequence to acquire a feature f₄¹; inputting the feature f₄¹ into the primary capsule layer of the capsule network in the first CMP module to acquire a vector f₄²; inputting the vector f₄² into the primary capsule layer of the capsule network in the first CMP module to acquire a feature f₄³; inputting the feature f₄³ into the first fully connected layer and the second ReLU activation function layer of the first CMP module in sequence to acquire a feature f₄⁴; and inputting the feature f₄⁴ into the second fully connected layer of the first CMP module to acquire a feature f(q″ᵢ);
j-3) inputting the feature vector μᵤ and the feature f(q″ᵢ) into the metric module of the few-shot classification model, and calculating the similarity score E_{w}(μᵤ, f(q″ᵢ)) by E_{w}(μᵤ, f(q″ᵢ))=∥μᵤ−f(q″ᵢ)∥; and
j-4) calculating a class label ŷᵢ of the i-th normalized ECG signal q″ᵢ by \vec{y}ᵢ=arg max {E_{w}(μ₁, f(q″ᵢ)), E_{w}(μ₂, f(q″ᵢ)), . . . , E_{w}(μᵤ, f(q″ᵢ)), . . . , E_{w}(μ_{N}, f(q″ᵢ))}, and combining class labels of all NQ normalized ECG signals to form the classification result.
